(19) United States
(12) Patent Application Publication
(10) Pub. No.: US 2010/0273741 A1
(43) Pub. Date: Oct. 28, 2010

(54) Apparatus and Methods for Adipose Tissue Detection

(76) Inventor: Antoine Assaf, Pianoro (IT)

Correspondence Address: Themis Law, 7979 Ivanhoe Ave Ste 400, La Jolla, CA, 92037, US

(21) Appl. No.: 12/742,517

(22) PCT Filed: Feb. 29, 2008
(86) PCT No.: PCT/IB2008/050747

§ 371 Date: May 12, 2010

(30) Foreign Application Priority Data
Nov. 14, 2007 (IT) VI2007A0000299

(51) Int. Cl. A61K 31/685; A61B 5/05; A61M 5/14; A61N 5/067; A61N 7/00; A61H 1/00; A61M 1/00; A61P 3/06

(52) U.S. Cl. 514/78; 600/430; 604/506; 607/89; 601/2; 601/1; 604/542

(57) ABSTRACT

The present invention finds application in the field of cosmetic medicine and particularly relates to an apparatus for adipose tissue detection which comprises a first electronic circuit for generating a beam of electromagnetic waves, radiating means for orienting the beam to an adipose tissue-containing part, sensor means for detecting reflected waves, a second electronic circuit for receiving the reflected electromagnetic waves, a unit for measuring a predetermined characteristic of the reflected waves and for producing an analog signal, a third electronic circuit for converting the analog signal into a digital signal, interface means providing an interface between the third electronic circuit and a graphic processing unit. The first electronic circuit comprises means for modulating the frequency of the generated waves, which operate in a microwave range from 1 GHz to 12 GHz.

### TECHNICAL FIELD

The present invention generally finds application in the field of cosmetic medicine and particularly relates to an apparatus and a cosmetic method for adipose tissues detection.

### BACKGROUND OF THE ART

It is known that, in cosmetic or therapeutic treatments such as liposculpture, lipodrainage and similar treatments for reducing and/or shaping adipose tissue in the human body, health operators need to assess as accurately as possible the amount and distribution of such tissue to define which part has to be removed.

Indeed, some adipose tissue is needed to allow body reshaping, otherwise, in case of insufficient or inadequate fat accumulation left by the operator, considerable skin corrugation would occur, leading to serious defects affecting beauty.

In the latter case, a second fat removal procedure would be required, with considerable apparent drawbacks for the patient.

Excessive adipose tissue removal would also cause considerable problems, causing hard-to-solve dermal trough problems.

Typically, this kind of assessment only relies on the manual sensitivity of the operator, and this obviously leads to the difficulty of determining with the utmost accuracy the amount of fat to be removed and especially not to be removed.

Bone and muscle detection techniques are widespread in the field of cosmetic and diagnostic medicine, which utilize the properties of electromagnetic radiation, and particularly of the waves of the radio-frequency range, such as X rays and y rays, or use ultrasound technologies.

Nonetheless, these methods are of no use for adipose tissue detection and in certain cases the energy associated with the wave beams would have relatively high values, and cause tissue destruction.

Liposuction methods are also known which utilize microwaves, as disclosed in U.S. Pat. No. 5,295,955, or high frequency radio waves directed against the adipose tissue for causing it to be softened and more easily removed in the next step using traditional instruments, such as a suction catheter.

Nevertheless, in addition to the potential dangers of the method, here again there is no way to immediately and accurately detect, before the procedure proper, the exact amount and distribution of the adipose tissue, leading to the above drawbacks.

### DISCLOSURE OF THE INVENTION

The object of the present invention is to overcome the above drawbacks, by providing an apparatus for detection of adipose tissue in the human body that is efficient and reliable.

A particular object is to provide an apparatus that allows for exact and relatively accurate assessment of the amount and distribution of adipose tissue or any lipid formation in the human body.

A further object is to provide an apparatus for detection of adipose tissue in the human body that is hand held and of simple and immediate use.

Yet another object is to provide an apparatus that is not invasive for patients undergoing a cosmetic or therapeutic treatment and has a high degree of safety.

Another important object is to provide a cosmetic method for detection of adipose tissue in the human body that allows for relatively accurate measurement of the distribution and amount of adipose tissue in a part of a human body, and imaging thereof as close as possible to reality.

These and other object, as better explained hereafter, are fulfilled by an apparatus according to the invention, which comprises a first electronic circuit for generating electromagnetic waves of predetermined frequency, radiating means for orienting the waves to an adipose tissue-containing part, sensor means for detecting reflected waves from the part being examined, a second electronic circuit for receiving and treating the reflected waves, a measuring unit connected to the second electronic circuit for measuring a predetermined characteristic of the reflected waves and for producing at least one analog control signal, a third electronic circuit for converting the analog signal into a digital signal and storing it, interface means providing an interface between the third circuit and a unit for graphic processing of the digital signal.

According to a peculiar feature of the invention, the first electronic circuit comprises frequency modulation means operating in a microwave range from 1 GHz to 12 GHz.

Thus, the transmitted and reflected microwaves can propagate through at least part of the adipose tissue possibly associated with the part under examination, to allow measuring thereof without causing structural alterations.

In another aspect, the invention relates to a method for adipose tissue detection.

In yet another aspect, the invention relates to a non therapeutic method for adipose tissue detection and reduction.

In yet another aspect, the invention relates to another method for adipose tissue reduction.

Advantageous embodiments of the apparatus and method of the invention are as defined in the dependent claims.

### BRIEF DESCRIPTION OF DRAWINGS

Further characteristics and advantages of the invention will be more readily apparent upon reading of the detailed description of a preferred non exclusive embodiment of an apparatus and a method for adipose tissue detection, a non therapeutic method and a surgical method for reduction of adipose tissue in the human body according to the invention, which are shown as non limiting examples with the help of the annexed figures, in which:

FIG. 1 is an exemplary schematic view of an apparatus of the invention;

FIG. 2 shows a block diagram of a method for adipose tissue detection according to the invention;

FIG. 3 shows a block diagram of a non therapeutic method for adipose tissue reduction according to the invention.

FIG. 4 shows a block diagram of a surgical method for adipose tissue reduction according to the invention.

### BEST MODE FOR CARRYING OUT THE INVENTION

Referring to the above figures, the apparatus of the invention, generally designated by numeral 1, may be used for detection of adipose tissue in one or more parts of a body.

Particularly, the apparatus 1 may be employed for detection of adipose tissue present in the muscular tissue to facilitate lipodrainage or liposculpture procedures, or linphodrainage treatments or the like.

The apparatus 1 may be also used for detecting lipid matter in the human vascular system or for finding tumorous masses within adipose tissues.

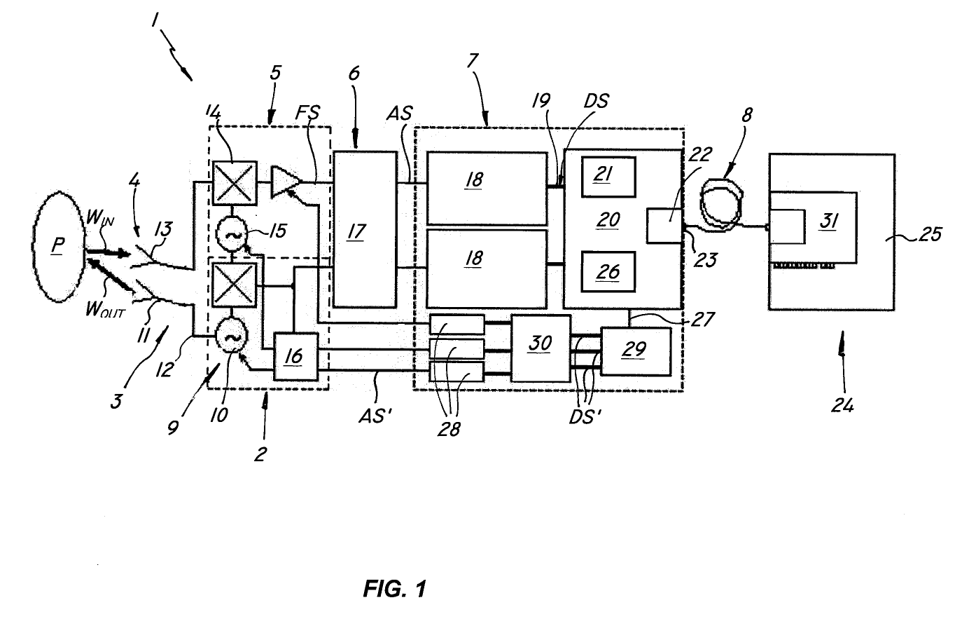

As shown in FIG. 1, an apparatus 1 of the invention comprises a first electronic circuit 2 for generating electromagnetic waves W_{OUT} of predetermined frequency, radiating means 3 for orienting the generated electromagnetic waves W_{OUT} to an adipose tissue-containing part, schematically indicated by P, and obtaining reflected waves W_{IN}, sensor means 4 for detecting the reflected waves W_{IN}, a second electronic circuit 5 for receiving the reflected waves W_{IN}, a measuring unit 6 connected to the second circuit 5 for measuring a predetermined characteristic associated with the reflected waves W_{IN} and for producing at least one analog control signal AS.

The latter is transmitted to a third electronic circuit 7 which converts it into a digital signal DS, to be stored by such third circuit 7, which is adapted to be connected via interface means 8 to a graphic processor or external computing unit for treatment and graphic processing of the stored digital signals DS.

According to a peculiar feature of the invention, the first electronic circuit 2 comprises means 9 for modulating the frequency of the generated electromagnetic waves W_{OUT}, which operate in a microwave range from 1 GHz to 12 GHz.

It was surprisingly found that such frequency values for the electromagnetic waves allow the latter to propagate through at least part of the adipose tissue and be reflected by a muscular tissue, without causing any physical alteration of the adipose tissue and leading to a substantially negligible absorption thereby.

Preferably, the modulation means 9 may be configured to produce output microwaves W_{OUT} from the first electronic circuit 2, having frequencies from 1 GHz to 6 GHz and preferably of about 3 GHz.

This is because it was surprisingly found that frequency values in the latter range produce minimized absorption by the adipose tissue, which ensures more reliable measurement.

In a preferred, non exclusive configuration of the invention, the radiating means 3 may include an electromagnetic wave generator 10 selected from the group comprising oscillators.

For example, a first voltage-controlled oscillator may be used, which is designated for clarity by the same numeral 10, of the low-power, dual-frequency adjustable type, with a frequency of 100 mW to 300 mW.

Nonetheless, all the parts described herein shall be intended as preferred technical choices, which can be replaced by any other technically equivalent and commonly available parts.

Particularly, an orientable scattering antenna 11, or similar scattering element may be provided at the output of the radiating means 3, for connection with the generator 10 via an insulating channel 12 to guide the generated microwaves W_{OUT}, the antenna 11 being preferably adapted to be oriented towards the part P to be examined.

The second electronic circuit 5 may in turn comprise a probe 13 adapted to be oriented towards the part P to be examined for receiving the reflected microwaves W_{IN}.

For instance, the probe 13, which is shown herein in schematic form, may be of coaxial type with a pair of cylindrical shields and a dielectric therebetween, such as Teflon or a similar material.

The shields have a free axial end which is susceptible of contacting the part P to be examined and an opposite axial end connected to the measuring unit 6. The probe 13 may be connected to a harmonic mixer 14 controlled by a second oscillator 15, preferably a voltage-controlled oscillator, which can be configured to generate less than 1 mW power.

The scattering antenna 11 and the receiving probe 13 may be integrated in a single part, which is adapted to transmit microwaves at the preset transmitted frequency and receive reflected waves at a frequency offset from the former.

The first 10 and second oscillators 15 may be coordinated by a further synchronizing circuit 16, such as a phase-looked loop commonly known as PLL, which will lock the frequency offset of the oscillators 10, 15 to an preset internal reference value, as is known in the art.

Also, the measuring unit 6 may include an I/O demodulator 17, which receives the frequency signal FS from the mixer 14 to measure the energy associated with the beam of reflected waves W_{IN} and generate one or more analog signals AS.

Energy measurement may occur, for instance, by measuring the effective amplitude of the reflected waves W_{IN}.

The third electronic circuit 7 may include a first converter board 18, or even more converter boards, of the ADC type, for converting the analog signals AS into corresponding digital signals DS. The first converter board 18 may be connected via a data input channel 19 to a memory board 20 that can be integrated in the third electronic circuit 7.

In one particular exemplary embodiment, the memory board 20 may be of the type commonly known as FEMCTRL, although any other type of functional equivalent board can be used.

The memory board 20 may have at least one first memory cell 21 for storage of one or more incoming digital signal DS and may be connected to the interface means 8. These latter may be integrated in the memory board 20 itself and essentially consist of an interface board 22 having a connection port 23 and a computing unit 24.

In one particular arrangement of the invention, the apparatus 1 may come with a preinstalled computing unit 24 connected to the connection port 23 of the memory board 20 for receiving a plurality of digital signals DS stored in the first memory cells 21 and processing a first set of data indicative of the quantity and distribution of adipose tissue in the part P being examined.

Particularly, the computing unit 24 may be a processor 25 for processing the first set of data, having a memory in which certain reference parameters are stored for comparison with the data of the first set and generating a second set of data susceptible of being graphically processed.

Furthermore, the computing unit 24 may be configured to generate a third set of data indicative of the frequency of scattered microwaves W_{OUT} which are transmitted to the memory board 20 to be stored in at least one second memory cell 26, conveniently dedicated therefor.

The latter cell is connected by a data output channel 27 to a second converter board 28 of the DAC type, possibly with the interposition of a serial-parallel converter 29 and a FIFO buffer 30, for converting the digital data DS′ of the third set into analog signals AS′ to be transmitted to the first circuit 2.

Also, the computing unit 24 may include means 31 for processing and/or graphically displaying the second set of data, selected from the group comprising monitors, printers and the like.

Thus, using suitable 2D or 3D graphic processing software, possibly of commonly available type, the adipose tissue may appear as close as possible to reality, in two- or three-dimensional form, thereby greatly facilitating the operations of the operator or surgeon.

In an alternative configuration of the invention, the apparatus 1 may be equipped with an internal computing unit 24 that can integrate one or more of the above parts, such as the memory board 20.

In any case, the computing unit 24 shall be capable of carrying out a test sequence, control the circuits for generating 2 and receiving 3 the waves W_{OUT} and W_{IN}, perform measurements and generate output reports, and shall be further equipped with an interface for connection to a display system or another computer.

The apparatus 1 will further have a power supply system, not shown, which may be a common battery but is preferably equipped with a stability control and one or more switches, such as FET transistors, for selective control of power supply to the various parts and possibly a backup battery.

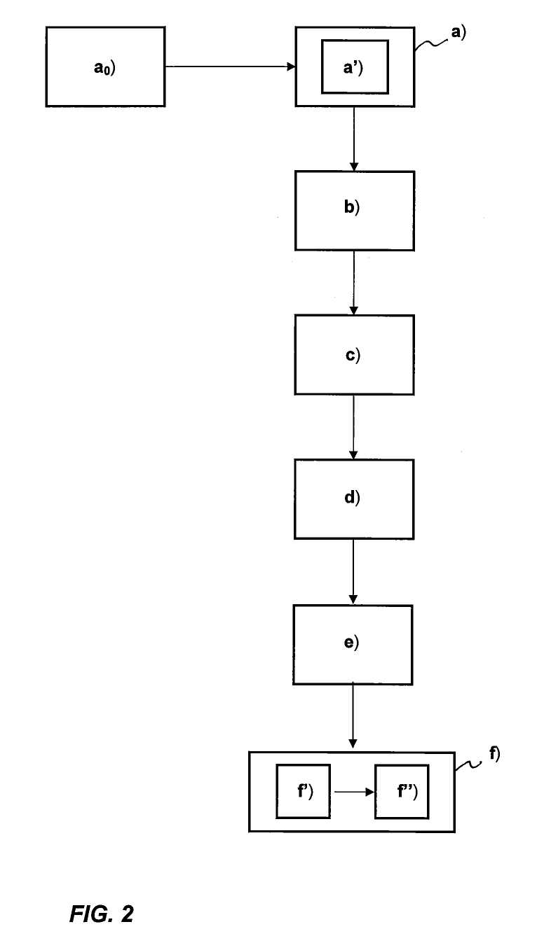

FIG. 2 schematically illustrates a cosmetic method for detection of adipose tissue in the human body, which can be carried out using the above apparatus, and comprises the steps of: a) of generating a beam of electromagnetic waves W_{OUT} of predetermined frequency, a step b) of radiating the generated waves W_{OUT} to a part P to be examined to obtain reflected waves W_{IN}, a step c) of measuring the amplitude of the reflected waves W_{IN} and generating an analog control signal AS.

The latter signal is then converted in the next step d) into a digital signal DS and transmitted to a computing unit 24 for comparison (step e)) with a reference value stored in the computing unit 21, which thus generates a first data set indicative of the amount and distribution of adipose tissue possibly associated with the part P being examined.

According to the invention, the waves W_{OUT} generated in step a) are modulated (step a′) within the range of microwaves having frequencies from 1 GHz to 12 GHz and preferably from 1 GHz to 6 GHz.

Once the first data set has been generated in step e), a step f) follows, for graphic processing of such data using a special computer or computing unit.

For example, the graphic processing step f) may include a first step f) of capturing an image of the part P to be treated, e.g. using a scanner, a camera or a similar device, adapted to be connected to a graphic processor, for displaying it on a screen and a step f″) of interpolation of the first data set to generate a plurality of level curves in the image, each indicating a quantitative value of the detected adipose tissue.

Furthermore, a calibration step a₀) may be provided upstream from the step a) of generating the waves W_{OUT}, to obtain one or more reference parameters with which the digital signals DS from step d) are to be compared for graphic processing.

For example, the calibration step a₀) may consist of the steps a) to e) as described above, to be carried out while directing the beam of waves W_{OUT} of known frequency to a part P in which no adipose tissue is known to be present with reasonable certainty, such as a biceps, thereby defining a reference value (zero level) for the subsequent signals.

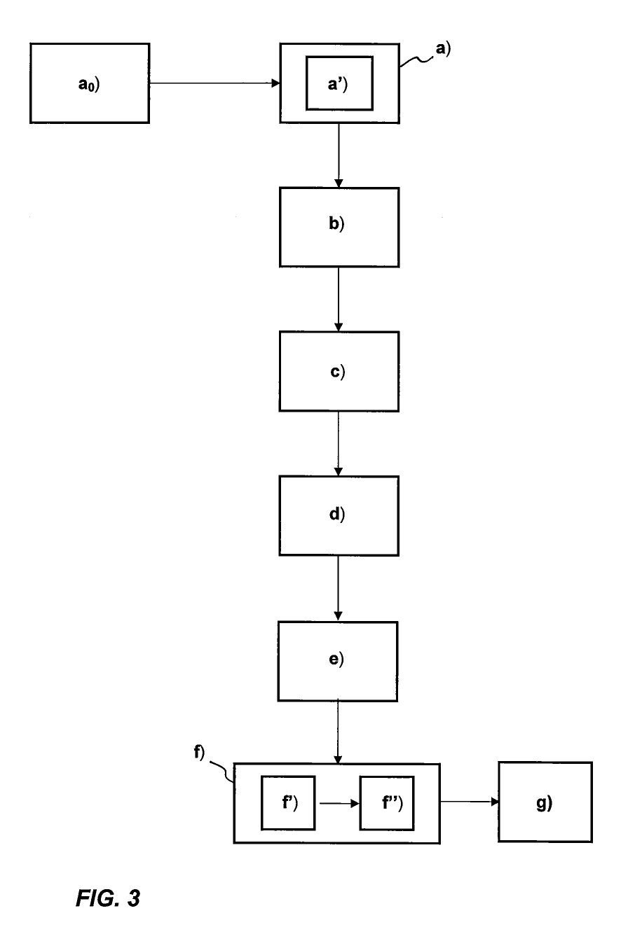

FIG. 3 schematically illustrates a non therapeutic method of the present invention for detection and reduction of adipose tissue in the human body, comprising the above steps from a) to f) and a step g) of treatment of the part P under examination for reduction of the adipose tissue associated therewith.

For example, the method may be used for cosmetic liporeduction by intradermal injection of a predetermined dose of a drug or mixture of drugs, particularly phosphatidyl choline or a mixture of drugs containing phosphatidyl choline, into the part P under examination.

Otherwise, the liporeduction treatment may be carried out by irradiating the part P under examination with a beam of electromagnetic waves whose frequency is modulated in the range of ultrasounds or infrared radiation, or with a laser beam, using known methods.

Also, the treatment may consist of a massage performed manually by a specialized operator or using equipment specially designed therefor.

The use of an apparatus 1 of the present invention for cosmetic or non therapeutic treatments as described above provides the apparent advantage of allowing both the operator and the patient undergoing such treatment, to immediately ascertain the effectiveness of the treatment.

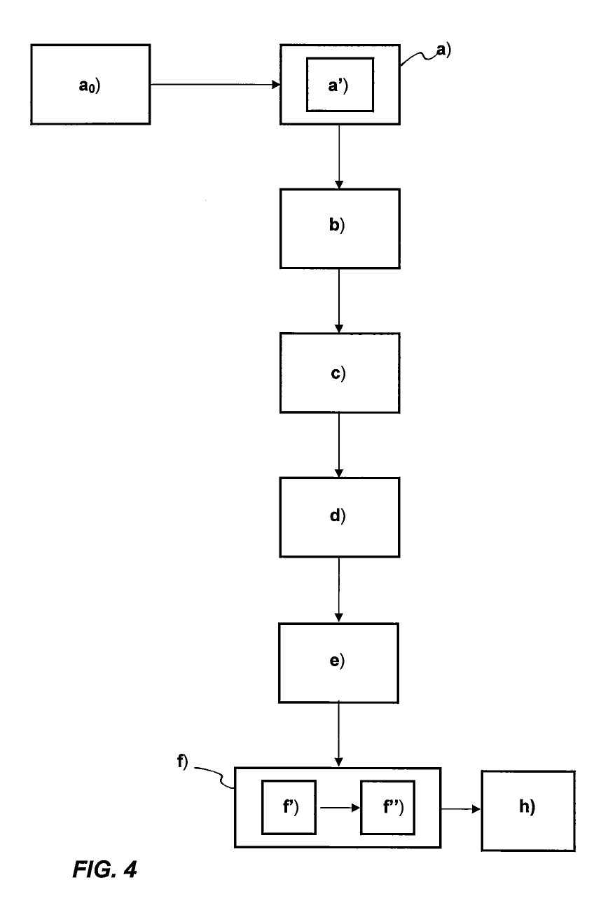

FIG. 4 schematically illustrates a surgical method for liporeduction of adipose tissues in a human body, comprising the above steps from a) to f) and a step h) of at least partially surgical reduction of the adipose tissue associated with the part P of the human body under examination.

Particularly, step h) may consist of a a liporeduction carried out according any of the commonly known surgical techniques, also of the invasive type such as liposuction by a cannula.

The apparatus and methods of the invention are susceptible of numerous changes and modifications within the inventive principle disclosed in the annexed claims. All the details thereof may be replaced by other technically equivalent parts, and the materials may vary depending on different needs, without departure from the scope of the invention.

While the apparatus and methods have been described with particular reference to the annexed figures, the numerals referred to in the disclosure and claims are only used for the sake of a better intelligibility of the invention and shall not be intended to limit the claimed scope in any manner.

What is claimed is:

1. An apparatus for detection of adipose tissue in the human body, comprising:
a first electronic circuit configured to generate a beam of electromagnetic waves (W_{OUT}) of predetermined frequency;
radiating means for orienting the beam of waves (W_{OUT}) to an adipose tissue-containing part, to obtain reflected waves (W_{IN});
sensor means for detecting the reflected waves (W_{IN});
a second electronic circuit configured to receive and process the electromagnetic waves (W_{IN}) detected by said sensor means;
a measuring unit connected to said second electronic circuit and configured to measure a predetermined characteristic associated with said reflected waves (W_{IN}) and to produce at least one analog control signal;
a third electronic circuit configured to convert said analog control signal into a digital signal and to store said digital signal; and
interface means providing an interface between said third electronic circuit and a graphic processing unit configured to process said digital signal,
wherein said first electronic circuit comprises means for modulating the frequency of the generated waves (W_{OUT}), in a microwave range from 1 GHz to 12 GHz, to allow the transmitted and reflected microwaves (W_{OUT}, W_{IN}) to propagate through at least part of the adipose tissue coupled to a part being examined.

2. The apparatus as claimed in claim 1, wherein said means for modulating are configured to generate microwaves (W_{OUT}) having frequencies from 1 GHz to 6 GHz.

3. The apparatus as claimed in claim 1, wherein said first electronic circuit includes an electromagnetic wave generator selected from the group comprising oscillators.

4. The apparatus as claimed in claim 3, wherein said radiating means comprise an orientable scattering antenna and an insulating channel configured to guide the generated microwaves (W_{OUT}) and to connect said antenna to said generator.

5. The apparatus as claimed in claim 1, wherein said second electronic circuit comprises a probe adapted to be oriented toward the part to be examined and to receive the reflected microwaves (W_{IN}).

6. The apparatus as claimed in claim 1, wherein said measuring unit includes an I/Q demodulator configured to measure an intensity of the reflected waves (W_{IN}) and generate said at least one analog signal.

7. The apparatus as claimed in claim 6, wherein said measuring unit is designed to measure an effective amplitude of the reflected waves (W_{IN}).

8. The apparatus as claimed in claim 6, wherein said third electronic circuit comprises at least one first converter board configured to convert said at least one analog signal into a corresponding digital signal, and a memory board connected, via a data input channel, to said at least one first converter board, said memory board having at least one first memory cell configured to store said at least one digital signal.

9. The apparatus as claimed in claim 8, wherein said interface means comprise an interface board connected to or integrated in said memory board and having a port for connection to an external computing unit.

10. The apparatus as claimed in claim 9, further comprising a computing unit connected to said connection port of said interface board and configured to receive a plurality of said digital signals stored in said at least one first memory cell and to process a first set of data indicative of quantity and distribution of the adipose tissue.

11. The apparatus as claimed in claim 10, wherein said computing unit comprises a processor configured to process said first set of data and to generate a second set of data susceptible of being graphically processed.

12. The apparatus as claimed in claim 10, wherein said computing unit is designed to generate a third set of digital data indicative of the frequency of the microwaves (W_{OUT}) to be generated, said memory board comprising at least one second memory cell configured to store said third set of digital data.

13. The apparatus as claimed in claim 12, wherein said third electronic circuit comprises at least one second converter board configured to convert the data of said third set into output analog signals to be transmitted to said first electronic circuit.

14. The apparatus as claimed in claim 9, wherein said computing unit includes means for one or more of processing or graphically displaying said second set of data, said one or more of processing or displaying means comprising an electronic appliance.

15. A method of detection of adipose tissue, comprising the steps of:
a) generating a beam of electromagnetic waves (W_{OUT}) of predetermined frequency;
b) radiating the beam of waves (W_{OUT}) to a part to be examined, to obtain reflected waves (W_{IN});
c) measuring the-an amplitude of the reflected waves (W_{IN}) and generating an analog control signal;
d) converting said analog control signal into a corresponding digital signal; and
e) comparing said digital signal with a reference value for generating a first data set indicative of an amount and distribution of the adipose tissue associated with the part being examined;
wherein the electromagnetic waves (W_{OUT}) generated in said generation step (a) are modulated within a range of microwaves having frequencies from 1 GHz to 12 GHz.

16. The method claimed in claim 15, further comprising a step (f) of graphical processing of said first data set using a computer.

17. The method as claimed in claim 16, wherein said graphic processing step (f) includes a step of capturing an image of the part being examined that is displayable on a screen, and a step of interpolating said first data set to generate a plurality of level curves in said image, each indicating a quantitative value of the detected adipose tissue.

18. The method as claimed in claim 15, wherein a radiation calibration step is provided upstream from the step (a), for steering the radiated beam (W_{OUT}) to a substantially adipose tissue-free part, to obtain said reference value.

19. A method of detection and reduction of adipose tissue in a human body, comprising the steps of:
a) generating a beam of electromagnetic waves (W_{OUT}) of predetermined frequency;
b) radiating the beam of waves (W_{OUT}) to a part of the human body to be treated, to obtain reflected waves (W_{IN});
c) measuring the amplitude of the reflected waves (W_{IN}) and generating a control signal;
e) comparing said control signal with a reference value such to generate a first data set indicative of amount and distribution of the adipose tissue associated with the part to be treated;
(f) graphically processing said first data set, using a computer, said electromagnetic waves (W_{OUT}) generated in said generation step (a) being modulated in the microwave range; and
g) treating the part being examined for reducing the adipose tissue associated therewith.

20. The method as claimed in claim 19, wherein said microwaves are modulated within the range of frequencies from 1 GHz to 12 GHz and preferably from 1 GHz to 6 GHz.

21. The method as claimed in claim 20, wherein said graphic processing step (f) includes a step of capturing an image of the part to be treated, said image being structured to be displayed on a screen, and a step of interpolation of said first data set to generate a plurality of level curves in said image, each indicating a quantitative value of the detected adipose tissue.

22. The method as claimed in claim 19, wherein said treatment step (g) is a liporeduction step, which is carried out by injecting a predetermined dose of a drug into the part of the human body to be treated.

23. The method as claimed in claim 22, wherein said drug is selected form the group consisting of phosphatidyl choline and mixtures of drugs comprising phosphatidyl choline.

24. The method as claimed in claim 19, wherein said treatment step (g) is a liporeduction step, which is carried out by irradiating said part of the human body with a beam of electromagnetic waves whose frequency is modulated in a range of ultrasounds or infrared radiation or with a laser beam.

25. The method as claimed in claim 19, wherein said treatment step (g) is a mechanical or manual massage of said part of the human to be treated.

26. A surgical method of liporeduction of adipose tissue in a human body, comprising the steps of:
a) generating a beam of electromagnetic waves (W_{OUT}) of predetermined frequency;
b) radiating the beam of waves (W_{OUT}) to a part of the human body to be treated, to obtain reflected waves (W_{IN});
c) measuring the amplitude of the reflected waves (W_{IN}) and generating a control signal;
d) comparing said control signal with a reference value for generating a first data set indicative of the amount and distribution of the adipose tissue associated with the part being examined;
(e) graphically processing said first data set, using a computer, said electromagnetic waves (W_{OUT}) generated in said generation step (a) being modulated in the microwave range and
f) at least partially surgically reducing the adipose tissue associated to the part of the human body being examined.

27. The method as claimed in claim 27, wherein said reduction step (f) is a liposuction.
